(19) United States
(12) Patent Application Publication
(10) Pub. No.: US 2023/0126661 A1
(43) Pub. Date: Apr. 27, 2023

(54) DENTAL FLOSSER

(71) Applicant: DenTek Oral Care, Inc., Tarrytown, NY (US)

(72) Inventors: Josef V. Knutzen, Allendale, NJ (US); Jessica Rica Lin, Bayside, NY (US)

(21) Appl. No.: 17/511,300

(22) Filed: Oct. 26, 2021

(51) Int. Cl. A61C 15/04; A61Q 11/00; A61K 8/02

(52) U.S. Cl. CPC A61C 15/046; A61Q 11/00; A61K 8/0204

(57) ABSTRACT

A dental flosser is provided for delivering to a mouth a moisture activated composition including a flavor agent. The dental flosser comprises a base portion forming a handle and terminating at a distal head portion. A pair of spaced apart arms extend from the head portion, each of the spaced apart arms having a proximal first end adjoining the head portion and a distal second end. A length of dental floss extends between the distal second ends of the arms. A surface of at least one arm of the pair of arms defines a cavity between the first end and the second end forming a well for accommodating the composition. The width of the cavity in a plane parallel to the surface of the at least one arm is substantially the same or greater than the width of the bottom of the cavity.

### BACKGROUND

A dental device is described and, more particularly, a dental flosser having structure for holding and delivering a composition, including a flavor agent or other active agent, to the mouth for yielding a desired or predetermined intra-oral activity. A method of making the dental flosser and loading the composition are also described.

Regular cleaning and flossing of the teeth are important for dental health. Beyond flossing, it is recognized that halitosis is another dental concern which may be helped by flavor agents. The desire for adding flavor agents to the mouth and teeth while flossing has led to the development of flavored coatings applied directly to the floss. In use, the flavored dental floss leaves a light flavoring in the mouth as the floss is passed between teeth. Unfortunately, the amount of flavor agent available and thus delivered into a user's mouth is small.

Dental floss holders, or “dental flossers”, have recently become a popular way of flossing between teeth. A typical dental flosser has two spaced arms across which is secured a strand of dental floss. Some dental flossers have openings or depressions for retaining flavor agent on the device itself. The openings are filled by using a mechanized system where miniature pistons pump flavor agent into the cavity via a heated nozzle system. The cavity is provided with at least one flange that serves as a mechanism to help retain the flavor agent within the cavity. However, the amount of flavor agent provided with this configuration is still small, and loading the flavor agent during manufacturing is difficult and inefficient.

For the foregoing reasons, there is a need for a dental flosser which is also effective for delivering a flavor agent or other active agents to the mouth. The dental flosser should retain the flavor agent or active agents in the dental flosser until used. Ideally, the dental flosser will enable cleaning in between teeth and delivery of sufficient flavor agent or other oral care agent to the mouth to provide teeth cleaning, breath freshening or other oral care benefits.

### SUMMARY

A dental flosser is provided for delivering to the mouth a moisture activated composition including a flavor agent. The dental flosser comprises a base portion forming a handle and terminating at a distal head portion. A pair of spaced apart arms extend from the head portion, each of the spaced apart arms having a proximal first end adjoining the head portion and a distal second end. A length of dental floss extends between the distal second ends of the arms. A surface of at least one arm of the pair of arms defines a cavity between the first end and the second end forming a well for accommodating the composition. The width of the cavity along its depth in a plane parallel to the surface of the at least one arm is substantially the same or greater than the width of the bottom of the cavity.

In one aspect, each of the pair of spaced apart arms of the dental flosser defines at least one cavity. The cavity may have a depth less than the thickness of the arm. More specifically, the cavity has width of about 2.75 mm wide, a depth of about 1.00 mm, and a length of about 5.77 mm.

In another aspect, the cavity has a central longitudinal axis and a slot in the bottom of the cavity extending along the central longitudinal axis of the cavity, the slot having a length less than the respective dimension of the cavity along the central longitudinal axis corresponding to the cavity length, the cavity length being greater than a respective dimension of the slot. More specifically, the slot has a depth of about 0.41 mm, a width of about 1.00 mm, and a length of about 4.03 mm.

A system of oral care is also provided, comprising a moisture activated composition comprising a flavor agent and a dental flosser. The dental flosser comprises a base portion forming a handle and terminating at a distal head portion. A pair of spaced apart arms extend from the head portion, each of the spaced apart arms having a proximal first end adjoining the head portion and a distal second end. A surface of at least one arm of the pair of arms defines between the first end and the second end a cavity having a width and forming a well for receiving the composition. The width of the cavity in a plane parallel to the surface of the at least one arm is substantially the same or greater than the width of the bottom of the cavity. A length of dental floss extends between the distal second ends of the arms.

In one aspect, the composition comprises materials selected from the group consisting of a flavor agent, an antimicrobial agent, a cleansing agent, a breath freshener, a mineralizing agent, a remineralizing agent, a therapeutic agent and combinations thereof. The cavity may be sized to accommodate from about 5 mm³ to about 15 mm³ of the composition.

In another aspect, each of the pair of spaced apart arms of the dental flosser defines at least one cavity. The cavity may have a depth less than the thickness of the arm. More specifically, the cavity has width of about 2.75 mm wide, a depth of about 1.00 mm, and a length of about 5.77 mm.

In a further aspect, the cavity has a central longitudinal axis and a slot in the bottom of the cavity extending along the central longitudinal axis of the cavity, the slot having a length less than the respective dimension of the cavity along the central longitudinal axis corresponding to the cavity length, the cavity length being greater than a respective dimension of the slot. More specifically, the slot has a depth of about 0.41 mm, a width of about 1.00 mm, and a length of about 4.03 mm.

A method of making a dental flosser is also provided. The method comprises the steps of providing a flosser comprising a base portion forming a handle and terminating at a distal head portion. A pair of spaced apart arms extend from the head portion, each of the spaced apart arms having a proximal first end adjoining the head portion and a distal second end. A length of dental floss extends between the distal second ends of the arms. A surface of at least one arm of the pair of arms defines at least one cavity between the first end and the second end forming a well. The width of the cavity in a plane parallel to the surface of the at least one arm is substantially the same or greater than the width of the bottom of the cavity. A liquid bath of a composition comprising a flavor agent is provided along with a base comprising a post extending from the base. The flosser is positioned over the bath, and the base advanced from a first position where the base is submerged in the bath to a second position such that the post extends vertically from the bath and registers with the cavity for delivery of flavor agent to the well. The base is retracted to the first position after the composition has cooled to a temperature at which the composition begins to solidify and bond to the walls of the cavity such that the composition is deposited in the well.

In one aspect, the distal end of the post is planar and has an area smaller than an area of the cavity defined in a plane at the surface of the arm.

In another aspect, the post is positioned centrally in the cavity.

### BRIEF DESCRIPTION OF THE DRAWINGS

For a more complete understanding of the dental flosser and method for making, reference should now be had to the embodiments shown in the accompanying drawings and described below. In the drawings:

FIG. 1 is a front perspective view of an embodiment of a dental flosser.

FIG. 2 is a rear perspective view of the dental flosser as shown in FIG. 1.

FIG. 3 is a right side elevation view of the dental flosser as shown in FIG. 1.

FIG. 4 is a left side elevation view of the dental flosser as shown in FIG. 1.

FIG. 5 is a front end elevation view of the dental flosser as shown in FIG. 1.

FIG. 6 is a rear end elevation view of the dental flosser as shown in FIG. 1.

FIG. 7 is a top plan view of the dental flosser as shown in FIG. 1.

FIG. 8 is a bottom plan view of the dental flosser as shown in FIG. 1.

FIG. 9 is a close-up front perspective view of a portion of the dental flosser as shown in FIG. 1 showing a flavor well.

FIG. 10 is a transverse cross-section view of the flavor well taken along line 10-10 of FIG. 3 of the dental flosser.

FIG. 11 is a front perspective view of a second embodiment of a dental flosser.

FIG. 12 is a rear perspective view of the dental flosser as shown in FIG. 11.

FIG. 13 is a right side elevation view of the dental flosser as shown in FIG. 11.

FIG. 14 is a left side elevation view of the dental flosser as shown in FIG. 11.

FIG. 15 is a front end elevation view of the dental flosser as shown in FIG. 11.

FIG. 16 is a rear end elevation view of the dental flosser as shown in FIG. 11.

FIG. 17 is a top plan view of the dental flosser as shown in FIG. 11.

FIG. 18 is a bottom plan view of the dental flosser as shown in FIG. 11.

FIG. 19 is a close-up front perspective view of a portion of the dental flosser as shown in FIG. 11 showing a flavor well.

FIG. 20 is a transverse cross-section view of the flavor well taken along line 20-20 of FIG. 13 of the dental flosser.

FIG. 21 is a front perspective view of a third embodiment of a dental flosser.

FIG. 22 is a rear perspective view of the dental flosser as shown in FIG. 21.

FIG. 23 is a right side elevation view of the dental flosser as shown in FIG. 21.

FIG. 24 is a left side elevation view of the dental flosser as shown in FIG. 21.

FIG. 25 is a front end elevation view of the dental flosser as shown in FIG. 21.

FIG. 26 is a rear end elevation view of the dental flosser as shown in FIG. 21.

FIG. 27 is a top plan view of the dental flosser as shown in FIG. 21.

FIG. 28 is a bottom plan view of the dental flosser as shown in FIG. 21.

FIG. 29 is a close-up front perspective view of a portion of the dental flosser as shown in FIG. 21 showing a flavor well.

FIG. 30 is a transverse cross-section view of the flavor well taken along line 30-30 of FIG. 23 of the dental flosser.

FIG. 31 is a longitudinal cross-section view of the flavor well taken along line 31-31 of FIG. 18 of the dental flosser.

FIG. 32 is a close up front elevation view of the dental flosser as shown in FIG. 3 with composition filling a cavity in each arm.

FIGS. 33-35 are top plan schematic view, side elevation and front elevation views, respectively, of an embodiment of an apparatus for delivering a fluid composition to a dental flosser.

FIG. 36 is a top perspective view of the delivery apparatus as shown in FIGS. 33-35 and a dental flosser in a first position.

FIG. 37 is a bottom perspective view of the delivery apparatus as shown in FIG. 36 and a dental flosser in a second loading position.

### DESCRIPTION

Certain terminology is used herein for convenience only and is not to be taken as a limiting. For example, words such as “upper,” “lower,” “left,” “right,” “horizontal,” “vertical,” “upward,” “downward,” “top” and “bottom” merely describe the configurations shown in the FIGs. Indeed, the components may be oriented in any direction and the terminology, therefore, should be understood as encompassing such variations unless specified otherwise. The words “interior” and “exterior” refer to directions toward and away from, respectively, the geometric center of the core and designated parts thereof. The terminology includes the words specifically mentioned above, derivatives thereof and words of similar import.

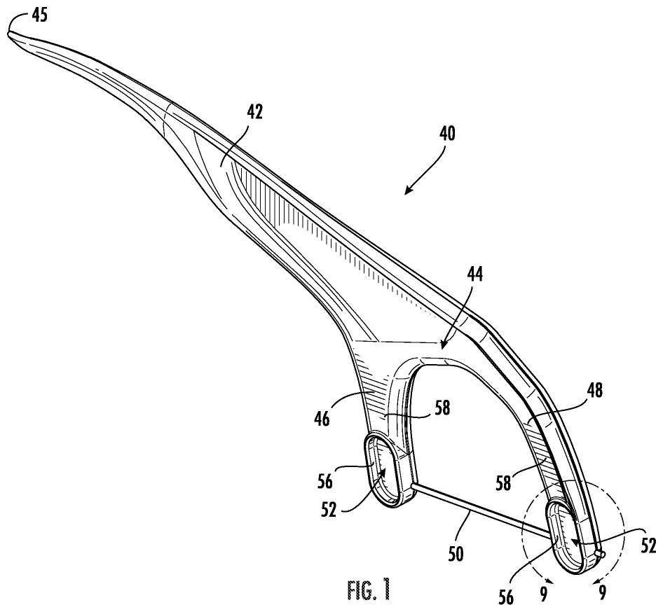

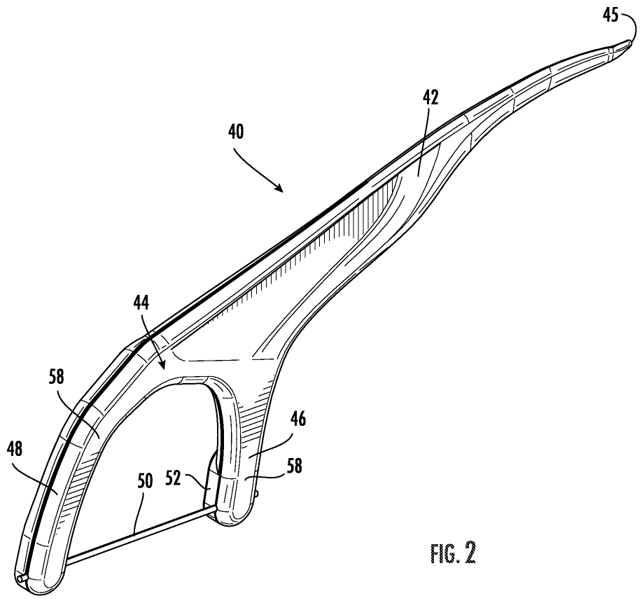

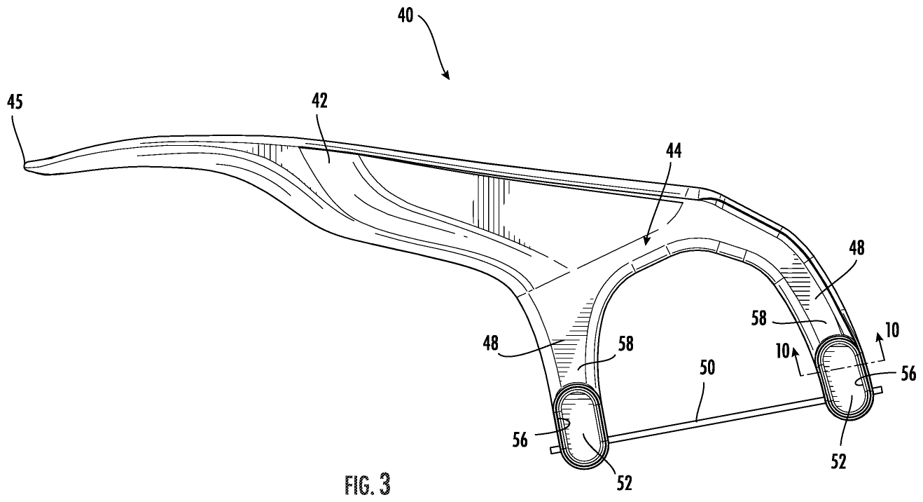

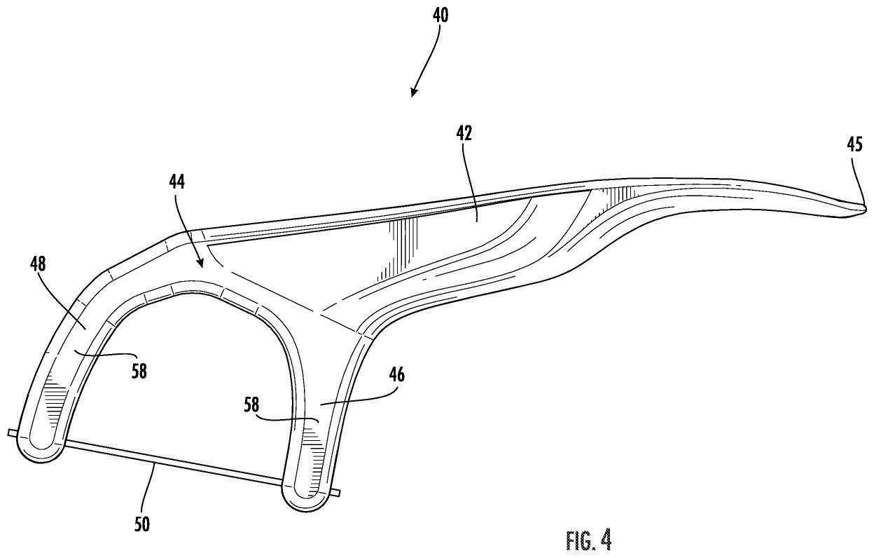

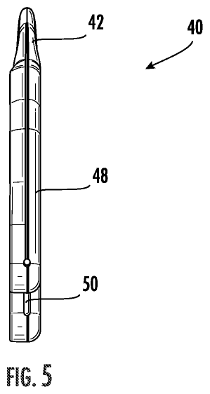

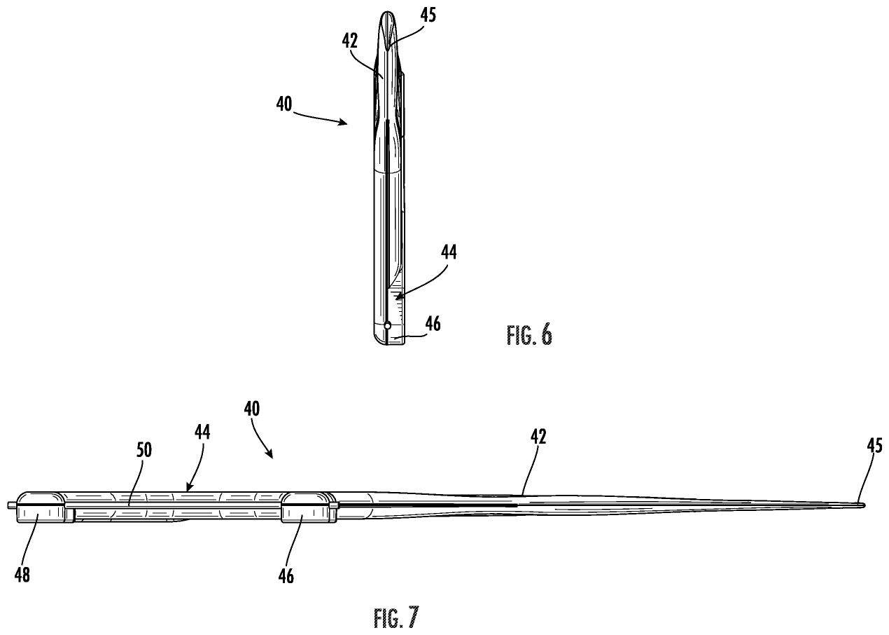

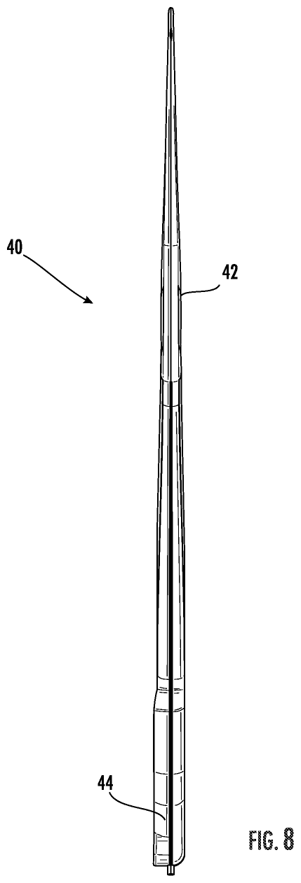

Referring now to the drawings, wherein like reference numerals refer to corresponding or similar elements throughout the several views, an embodiment of a dental flosser is shown in FIGS. 1-8 and generally designated at 40. The dental flosser 40 comprises a handle 42 terminating at a distal head 44 which is co-planar with the handle. A pair of spaced elongate arms 46, 48 extend from the head 44 forming a C-shaped opening. A length of dental floss 50 is secured between the distal ends of the arms 46, 48 for cleaning between teeth. The arms 46, 48 are shown oriented at a slight angle relative to one another, but it will be understood that the arms may be otherwise oriented so as to be parallel as long as the arms provide spaced apart ends for supporting the dental floss 50.

At least one of the arms 46, 48 defines a cavity 52 for receiving and retaining a composition 54 comprising a flavor agent or other oral active agent loaded therein. The cavity 52 may be in the form of a concavity or a through hole, either of which is capable of containing compositions having flavor agent or other agent. The composition 54 may be moisture-activated, and is held within the cavity 52 until the dental flosser 40 is used. In use during flossing, the composition 54 is contacted by saliva from the user's mouth and disperses throughout the mouth. Although one cavity 52 is shown in each arm 46, 48, it is understood that only one arm may have the cavity or either arm may have one or more cavities defined therein on one or both surfaces of the arms 46, 48.

The handle 42, head 44 and arms 46, 48 comprising the dental flosser 40 may be a single, unitary piece made of any suitable material known in the art. Suitable materials include polymers such as, but not limited to, polystyrene (especially high impact polystyrene), acrylics, such as poly methyl methacrylate; polyolefins, such as polyethylene and polypropylene; polyesters, such as polycaprolactone; co-polyesters; polycarbonate; and mixtures thereof. The dental flosser 40 is preferably made by injection molding, with the one or more cavities 52 being formed during the molding process. Injection molding of the device is preferred, but the dental flosser 40 may be made by any suitable process known in the art.

The dental floss 50 used in the dental flosser 40 may be any material known in the art. Examples of suitable dental floss material include, but are not limited to, a monofilament fiber or a multi-filament yarn comprising a plurality of such monofilament fibers. The dental floss 50 may be circular in cross-section or flat in cross-section, and typically has a denier ranging from about 200 to about 1400. The denier of the individual filaments forming the yarn typically ranges from about 1 to about 6, although other deniers may be used in some circumstances if desired. The yarn may be twisted or untwisted, as well as coated with substances to prevent fraying and provide ease of sliding between teeth. Psuedo-monofilament yarns may also be used as dental floss material. In one suitable embodiment, ultrahigh molecular weight polyethylene floss having a denier of 450 and 240 strands at 200 twists per meter is used.

The floss 50 is permanently attached to the arms 46, 48 in the molding process. Any suitable conventional process of manufacturing dental floss holders may be used to secure the floss 50 between the arms 46, 48, including injecting plastic into the mold to form the dental flosser 40 and then cutting and tying the ends of the floss. Alternatively, the ends of the arms 46, 48 are heated to form spheres and retain the floss material in place.

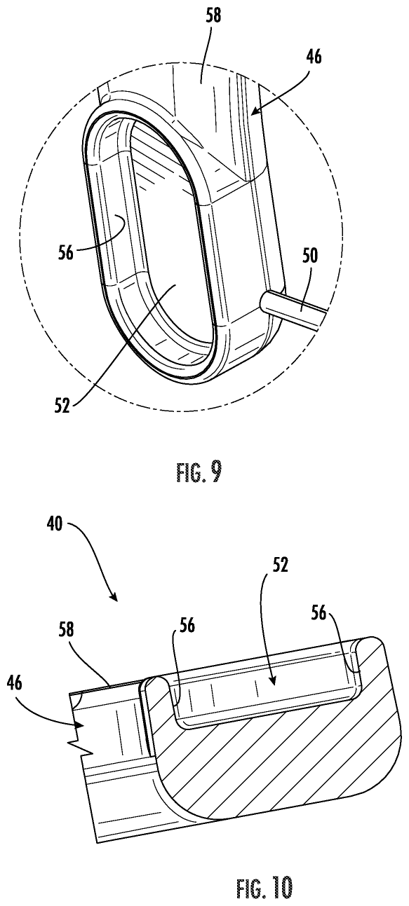

The dental flosser 40 is provided with at least one cavity 52 preferably located in a surface of at least one of the spaced-apart arms 46, 48 (FIGS. 9 and 10). Each cavity 52 can be in the form of a concave area or indentation that extends only partially through the arm 46, 48 and is open to only one side surface of the arm, and thus closed to an opposite side surface of the arm. Alternatively, the cavity 52 may be a through hole that extends completely through the arm (not shown). The walls of the arm 46, 48 defining the through hole must present enough surface area to hold the composition during loading, as will be described below. The cavity 52 defines a volume that is sufficient to accommodate an effective amount of the composition 54, such as a flavor agent.

The drawings depict the cavity 52 in the form of an oval, however, it is understood that the cavity may be a square, circle, triangle or any other suitable shape. The cavity 52 can be varied in size so as to accommodate varying volumes of the composition 54 for delivery during use of the dental flosser 40. In one example, the cavity 52 holds about 5 mm³ to about 15 mm³ of the composition. Each cavity 52 is defined by opposed side walls 56 which define a width or length of the cavity 52 therebetween. The opposite longitudinal side walls 56 may be substantially parallel as the cavity extends into the thickness of the arm. Near the surface of the arms 46, 48, the side walls 56 taper outwardly at an oblique angle with respect to an outer surface 58 of the arm 46, 48 such that the cavity 52 is widest at the surface of the arm. Thus, the width of the cavity 52 either remains the same or increases throughout the thickness of the arm 46, 48 from the bottom of the cavity to an opening at the surface 58 of the arm.

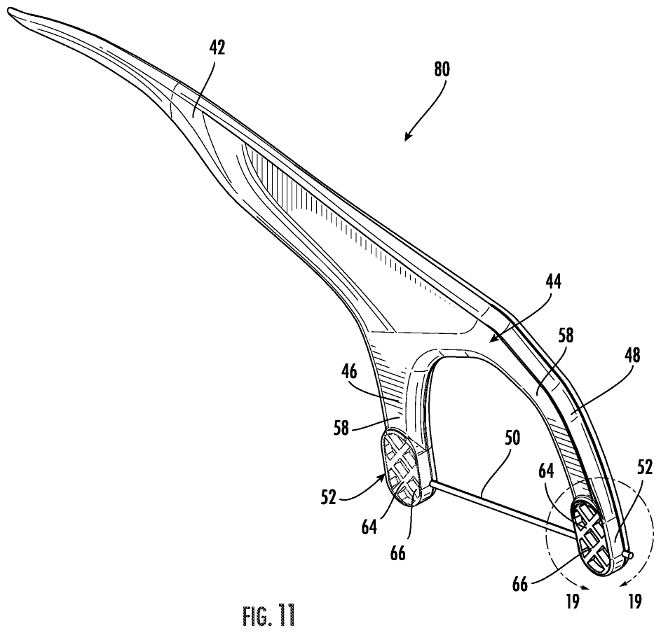

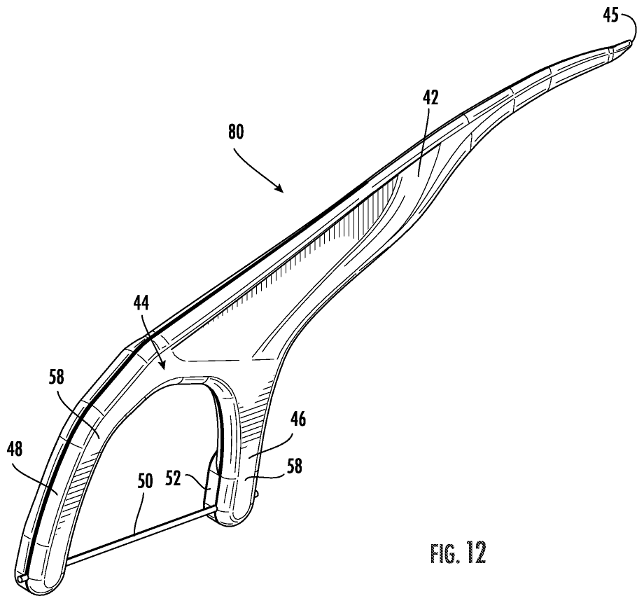

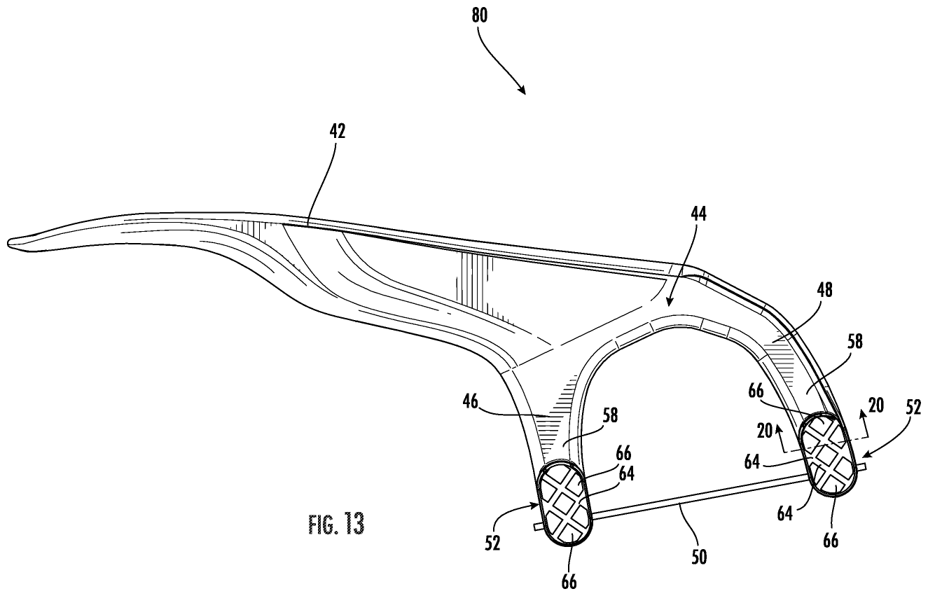

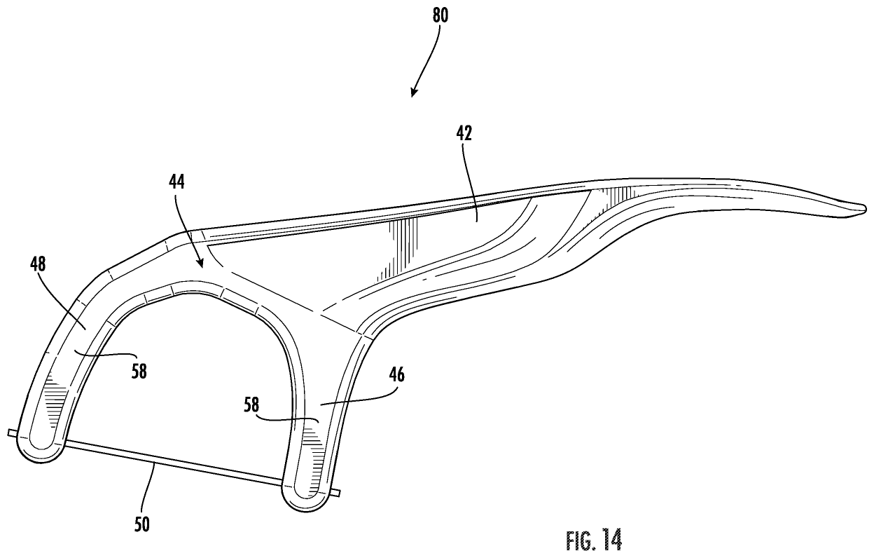

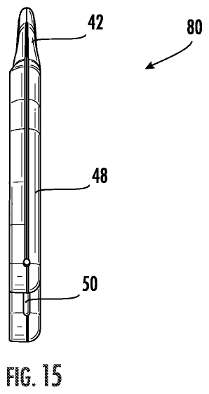

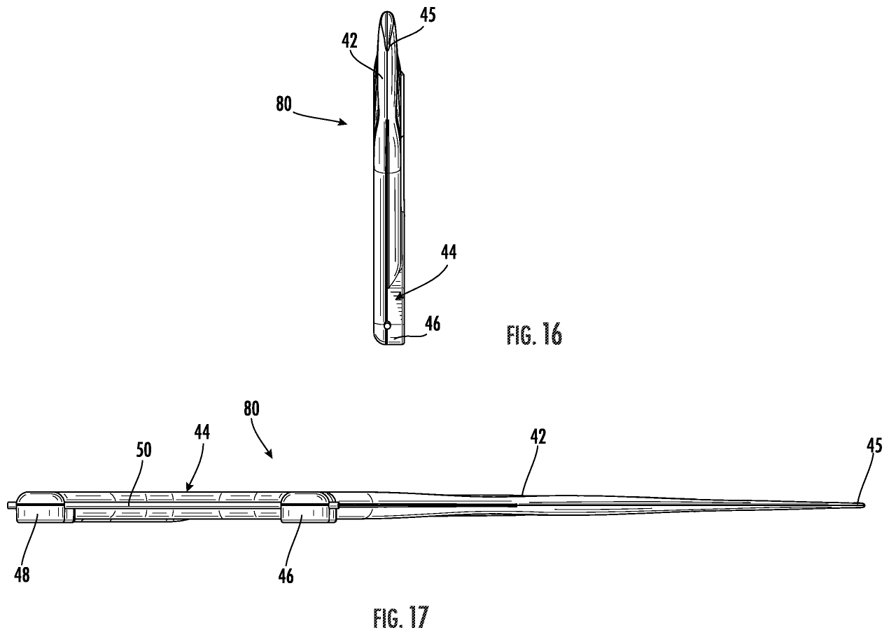

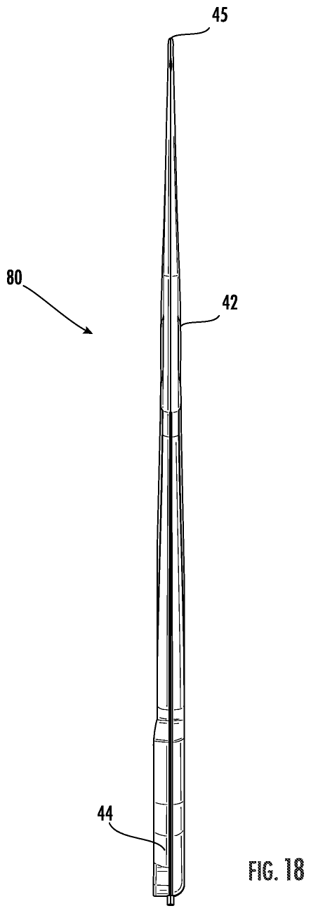

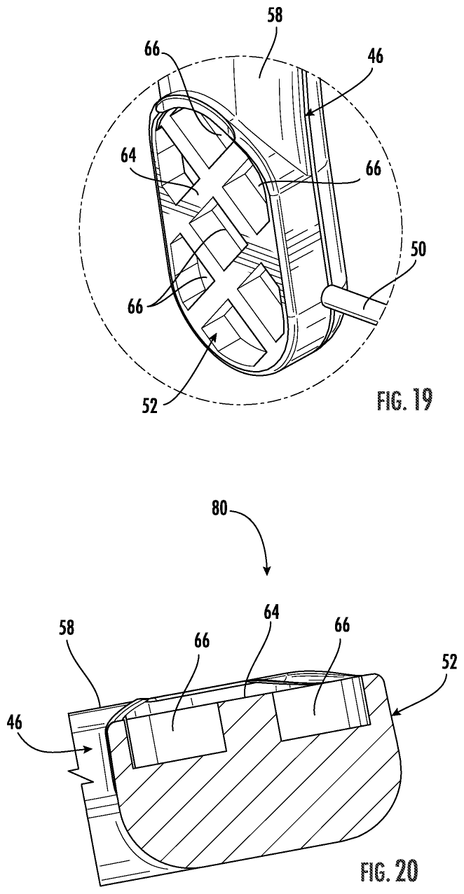

In an embodiment of the dental flosser shown in FIGS. 11-18, and generally designated at 80, each cavity 52 may further comprise a plurality of interconnected walls 64 extending between the side walls 56. The walls 56, 64 thereby form a pattern of separate wells 66 in the cavity (FIGS. 19 and 20). This configuration of the cavity 52 holds about 5 mm³ to about 9 mm³ of the composition.

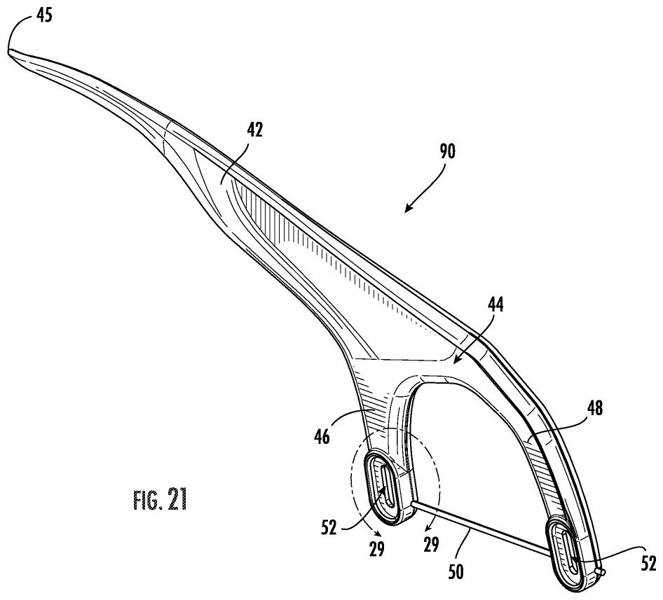

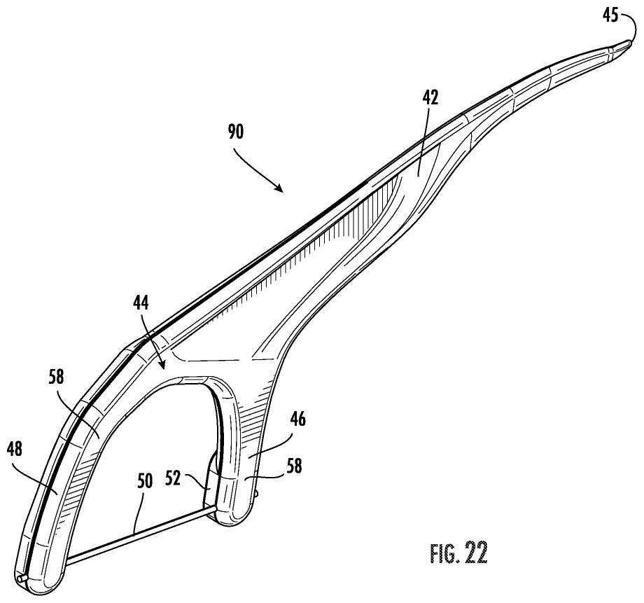

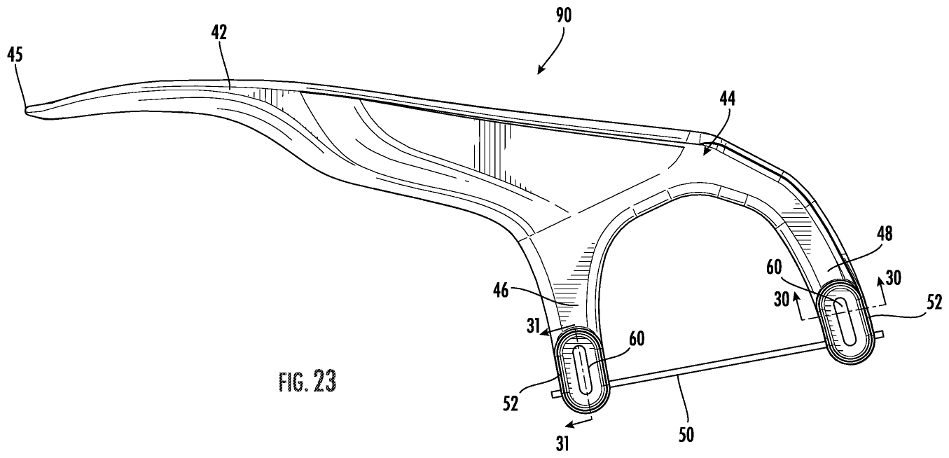

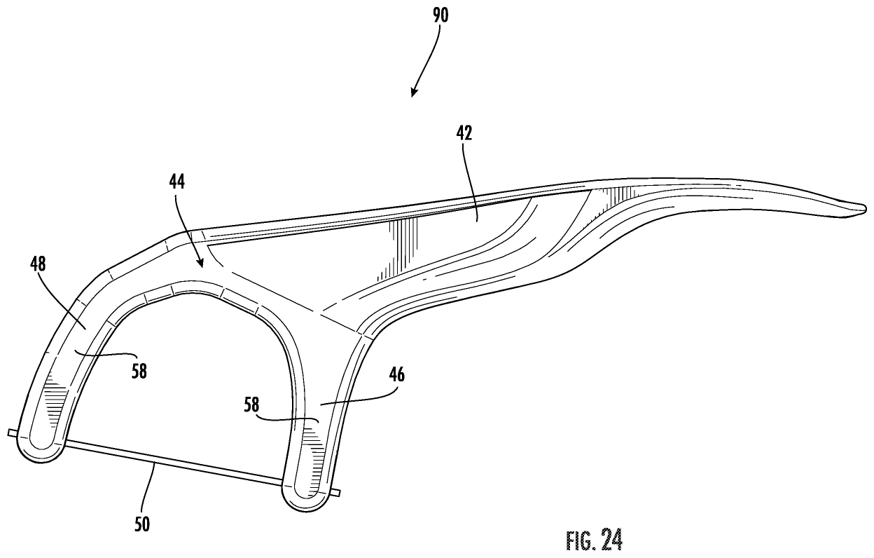

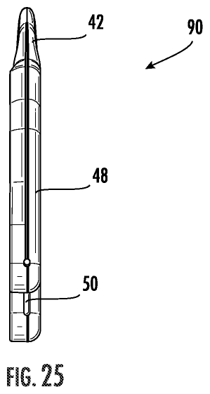

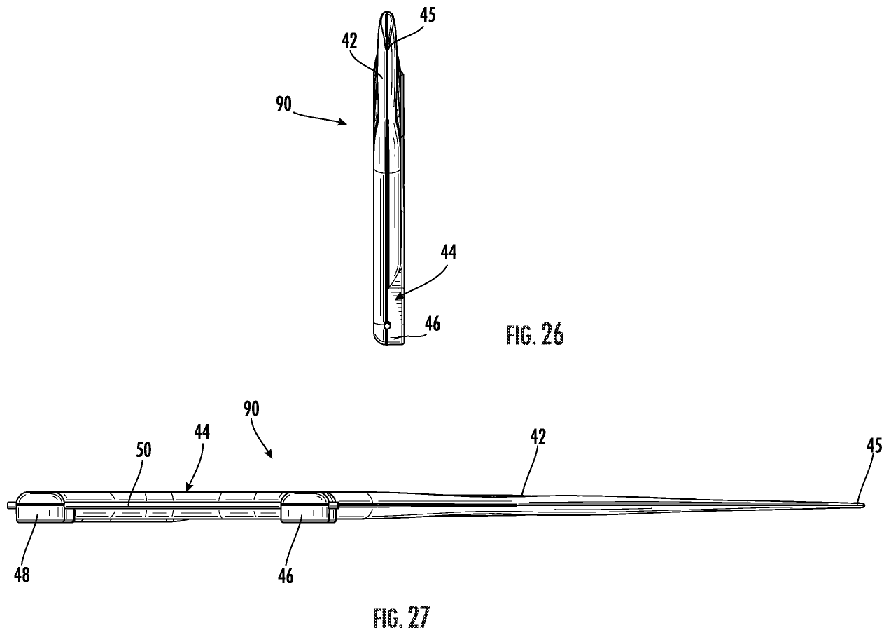

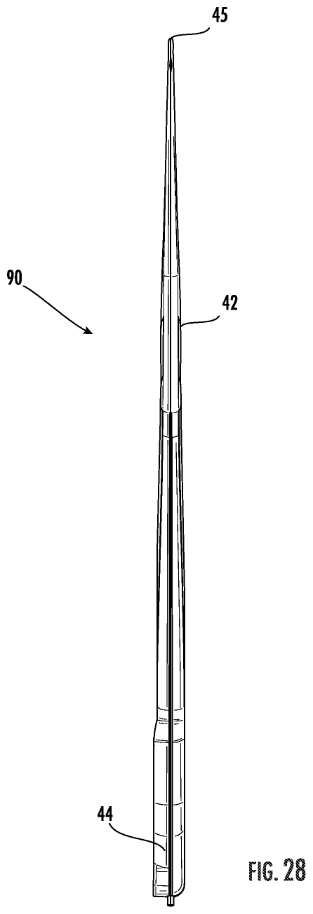

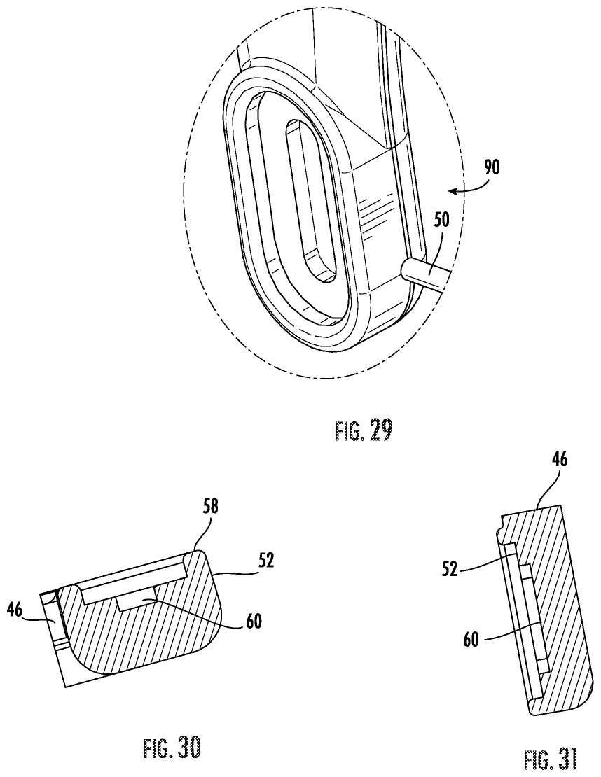

In another embodiment of the dental flosser shown in FIGS. 21-28, and generally designated at 90, a cavity 52 may comprise a central longitudinal slot 60. The slot 60 has a length less than the longitudinal length of the cavity 52 (FIG. 29). This configuration creates a step 62 in the cavity 52 such that the cavity has an outer portion along the depth of the cavity 52 having increased open area (FIGS. 30 and 31). Each cavity is approximately 2.75 mm wide, by about 1.00 mm deep and about 5.77 mm long, of which the slot is about 0.41 mm deep and about 1.00 mm wide and 4.03 mm long. Each cavity is sized to accommodate about 10-15 mm³ of the composition 54. It is understood that the cavities may be of uniform or non-uniform height, length, and width.

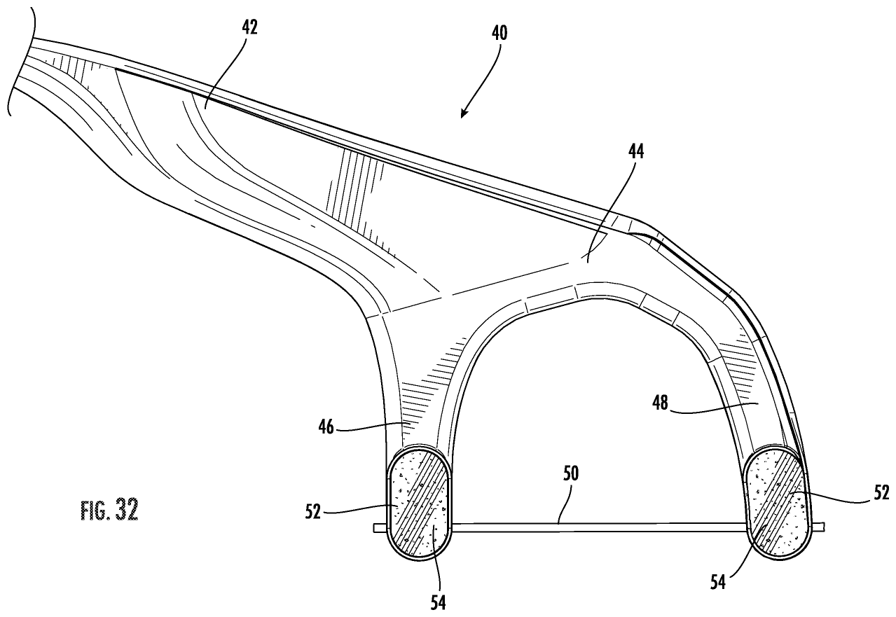

The composition 54 is capable of being contained within the cavity 52 and activated by saliva when in use during flossing (FIG. 32). In one embodiment, the composition 54 comprises a flavor agent in the form of a solid or semi-solid material when at room temperature. The flavor agent may be in the form of a water soluble or water insoluble composition that is capable of being filled into the cavity of the dental device. The water soluble composition may include, but is not limited to, polyethylene glycols (“PEG”), such as PEG 1000 to PEG 5000, ethylene oxide/propylene oxide copolymers, such as those commercially available under the trade name PLURONIC®, and polyvinyl alcohols. The composition may further comprise sweeteners, such as sucralose or acesulfame potassium, and xylitol, and other excipients to form a stable solid or semi-solid composition. The flavor agents may include, but are not limited to, menthol, sodium saccharin, sorbitol, aspartame, or other natural and artificial flavors. Breath fresheners may also be added to the composition, for example parsley seed, methyl salicylate, sunflower oils and peppermint oil. The dental flosser 40 can be provided to the user in different flavors. The composition may also contain moisturizing agents, lubricating agents, effervescent agents, sensory agents, soothing agents, anti-inflammatory agents and the like and combinations thereof.

Other suitable active agents related to oral care could be dispensed from the dental flosser 40. The oral activity provided by other active agents may be medical treatment, such as fluoridization, remineralization or mineralization and desensitization or aesthetic treatment such as tooth whitening or providing breath fresheners, or any other desired activity. Examples of hygiene useful material include fluoride, antibacterial agents, whitening agents, anti-inflammatory agents, anti-attachment agents, wax and felt. Such products include, but are not limited to, toothpaste, tooth powder or could be a small vial of mouthwash having a gel, a powder or a liquid. Alternatively, the composition can serve as a tooth whitening or bleaching agent selected from the group consisting of hydrogen peroxide, carbamide peroxide metal chlorites, perborates, percarbonates, peroxyacids, persulfates, urea peroxide, calcium peroxide, calcium chlorite, barium chlorite, magnesium chlorite, lithium chlorite, sodium chlorite, potassium chlorite, hypochlorite, chlorine dioxide, sodium percarbonate, oxones, and protease.

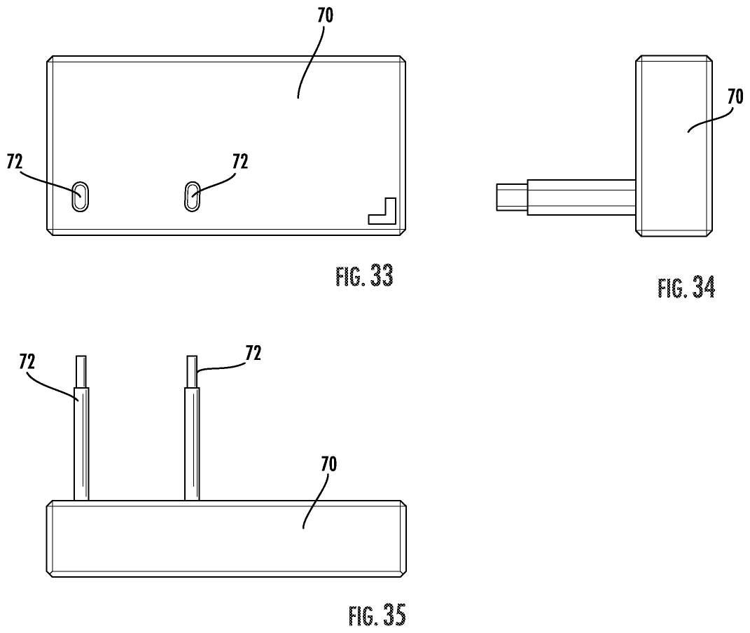

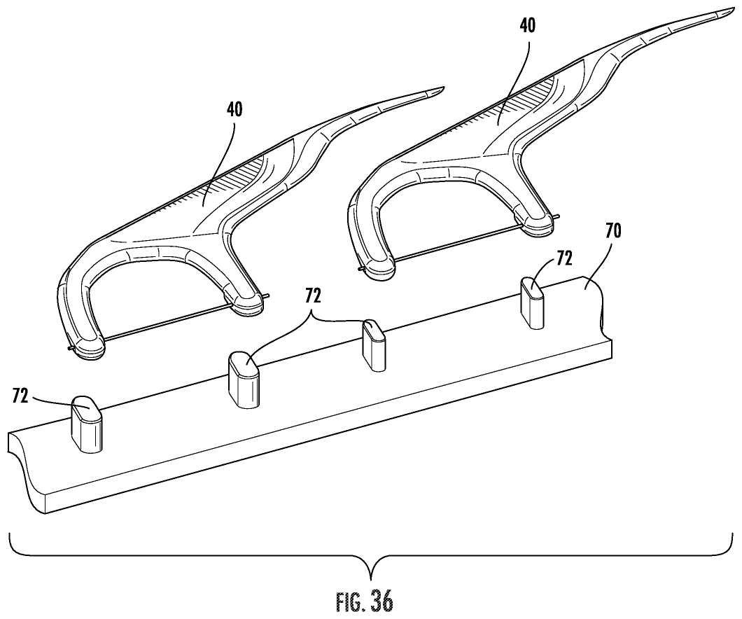

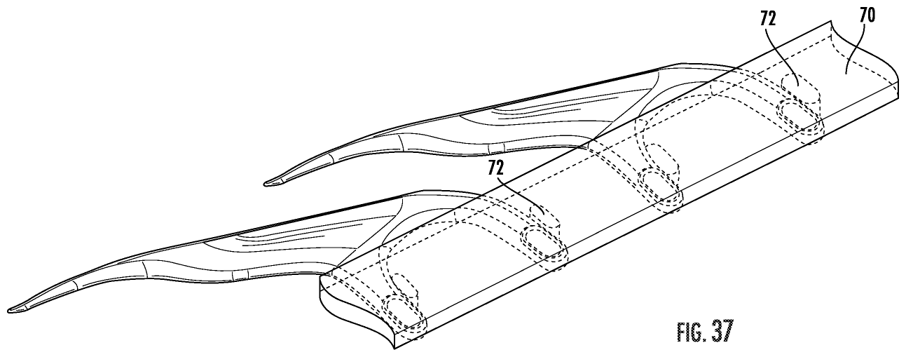

In the making of the dental flosser 40, 80, 90 a bath of the liquid composition 54 is heated to about 65° C. The dental flosser 40, 80, 90 is stationed over the bath. A “flavor jig” 70 (FIGS. 33-35) having two spaced posts 72 or pins is submerged in the bath in a first position shown in FIG. 36. Each post 72 corresponds to a cavity 52 on each arm 46, 48 of the dental flosser 40, 80, 90. The flavor jig 70 is raised out of the bath advancing the posts 72 in a vertical orientation to adjacent the cavities 52 in a second position shown in FIG. 37. Shown from below, the composition 54 on the ends of the posts 72 fills each cavity 52. The flavor jig 70 and pins 72 remain in the second loading position for about 1-2 seconds during which the composition 54 cools causing the composition to begin to solidify and bond to the dental flosser 40. The surface area of the cavity 52 helps with heat transfer for cooling the melted composition 54, as well as providing surface area for adhesion. The cavity 54 then retains the composition 54 within the cavity after the posts 72 retreat with the flavor jig 70 back to the first position. The cooled and solidified composition 54 is not easily prematurely dislodged from the cavity 52 prior to use until such time as the dental flosser 40 is placed within the mouth. In an embodiment wherein the cavity 52 is a through hole, a cooling plate (not shown) can be disposed against the top side of the flosser 40, 80, 90 during deposition of the composition. The plate cools the composition on contact and prevents the composition from flowing out of the opposite side of the through hole. The cooling plate can then be removed leaving the composition in the through hole while the flavor jig 70 retracts.

In use, the arms of the dental flosser 40, 80, 90 supporting the dental floss 50 are placed in the mouth to begin flossing. To floss the teeth, the user may position the floss 50 between the teeth and gently slide the floss up and down, allowing the floss to wrap around the teeth, cleaning above and below the gum line. Once the dental flosser 40, 80, 90 is inserted into the mouth and saliva contacts the composition 54 stored in the cavity 52, the composition 54 comprising the flavor agent or other active agent disperses throughout the user's mouth. The saliva activates the flavor system to deliver a strong flavor perception.

The dental flosser 40, 80, 90 has many advantages, including providing a significant and continuous flavor delivery throughout the mouth during flossing. The amount of composition 54 delivered is greater and, as a result, longer lasting than any known dental flosser. The dental flosser provides teeth cleaning and breath freshening, all of which deliver a cleaning, polishing, whitening, between teeth cleaning, and breath freshening action in addition to enhancing the cleaning efficiency of a typical disposable flosser.

Although the dental flosser and method of making have been shown and described in considerable detail with respect to only a few exemplary embodiments thereof, it should be understood by those skilled in the art that we do not intend to limit ourselves to the embodiments since various modifications, omissions and additions may be made to the disclosed embodiments without materially departing from the novel teachings and advantages of the apparatus, particularly in light of the foregoing teachings. Accordingly, we intend to cover all such modifications, omission, additions and equivalents as may be included within the spirit and scope of the apparatus, system and method as defined by the following claims. In the claims, means-plus-function clauses are intended to sticker the structures described herein as performing the recited function and not only structural equivalents but also equivalent structures. Thus, although a nail and a screw may not be structural equivalents in that a nail employs a cylindrical surface to secure wooden parts together, whereas a screw employs a helical surface, in the environment of fastening wooden parts, a nail and a screw may be equivalent structures.

What is claimed is:

1. A dental flosser for delivering to a mouth a moisture activated composition including a flavor agent, the dental flosser comprising:
a base portion forming a handle and terminating at a distal head portion;
a pair of spaced apart arms extending from the head portion, each of the spaced apart arms having a proximal first end adjoining the head portion and a distal second end;
a length of dental floss extending between the distal second ends of the arms,
wherein a surface of at least one arm of the pair of arms defines a cavity between the first end and the second end and extending into the thickness of the arm forming a well for accommodating the composition, and
wherein the width of the cavity in a plane parallel to the surface of the at least one arm is substantially the same or greater than the width of the bottom of the cavity.

2. The dental flosser as recited in claim 1, wherein the cavity has a depth less than the thickness of the arm.

3. The dental flosser as recited in claim 1, wherein each of the pair of spaced apart arms defines at least one cavity.

4. The dental flosser as recited in claim 1, wherein the cavity has width of about 2.75 mm wide, a depth of about 1.00 mm, and a length of about 5.77 mm.

5. The dental flosser as recited in claim 1, wherein the cavity has a central longitudinal axis and a slot in the bottom of the cavity extending along the central longitudinal axis of the cavity, the slot having a length less than the respective dimension of the cavity along the central longitudinal axis corresponding to the cavity length, the cavity length being greater than a respective dimension of the slot.

6. The dental flosser as recited in claim 5, wherein the slot has a depth of about 0.41 mm, a width of about 1.00 mm, and a length of about 4.03 mm.

7. A system of oral care comprising:
a moisture activated composition comprising a flavor agent;
a dental flosser comprising
a base portion forming a handle and terminating at a distal head portion,
a pair of spaced apart arms extending from the head portion, each of the spaced apart arms having a proximal first end adjoining the head portion and a distal second end, a surface of at least one arm of the pair of arms defining between the first end and the second end a cavity having a width and forming a well for receiving the composition, the width of the cavity in a plane parallel to the surface of the at least one arm substantially the same or greater than the width of the bottom of the cavity; and
a length of dental floss extending between the distal second ends of the arms.

8. The oral care system as recited in claim 7, wherein the composition comprises materials selected from the group consisting of a flavor agent, an antimicrobial agent, a cleansing agent, a breath freshener, a mineralizing agent, a remineralizing agent, a therapeutic agent and combinations thereof.

9. The oral care system as recited in claim 7, wherein the cavity is sized to accommodate from about 5 mm³ to about 15 mm³ of the composition.

10. The oral care system as recited in claim 7, wherein the cavity has a depth less than the thickness of the arm.

11. The oral care system as recited in claim 7, wherein the cavity has width of about 2.75 mm wide, a depth of about 1.00 mm, and a length of about 5.77 mm.

12. The oral care system as recited in claim 7, wherein each of the pair of spaced apart arms defines at least one cavity.

13. The oral care system as recited in claim 7, wherein the cavity has a central longitudinal axis and a slot in the bottom of the cavity extending along the central longitudinal axis of the cavity, the slot having a length less than the respective dimension of the cavity along the central longitudinal axis corresponding to the cavity length, the cavity length being greater than a respective dimension of the slot.

14. The oral care system as recited in claim 13, wherein the slot has a depth of about 0.41 mm, a width of about 1.00 mm, and a length of about 4.03 mm.

15. A method of making a dental flosser, the method comprising the steps of:
providing a flosser comprising
a base portion forming a handle and terminating at a distal head portion,
a pair of spaced apart arms extending from the head portion, each of the spaced apart arms having a proximal first end adjoining the head portion and a distal second end, and
a length of dental floss extending between the distal second ends of the arms,
wherein a surface of at least one arm of the pair of arms defines at least one cavity between the first end and the second end forming a well, and
wherein the width of the cavity in a plane parallel to the surface of the at least one arm is substantially the same or greater than the width of the bottom of the cavity;
providing a liquid bath of a composition comprising a flavor agent;
providing a base comprising a post extending from the base;
positioning a flosser over the bath;
advancing the base from a first position where the base is submerged in the bath to a second position such that the post extends vertically from the bath and registers with the cavity for delivery of flavor agent to the well; and
retracting the base to the first position after the composition has cooled to a temperature at which the composition begins to solidify and bond to the walls of the cavity such that the composition is deposited in the well.

16. The method of making a dental flosser as recited in claim 15, wherein the distal end of the post is planar and has an area smaller than an area of the cavity defined in a plane at the surface of the arm.

17. The method of making a dental flosser as recited in claim 15, wherein the post is positioned centrally in the cavity.
